(19) United States
(12) Patent Application Publication
(10) Pub. No.: US 2004/0009496 A1
(43) Pub. Date: Jan. 15, 2004

(54) Composition for bonding nucleic acid to a solid phase

(75) Inventors: Anja Eiblmaier, Wolfratshausen (DE); Elke Helftenbein, Stuttgart (DE)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: Antigene Biotech GmbH, Stuttgart (DE)

(21) Appl. No.: 10/325,395

(22) Filed: Dec. 19, 2002

(30) Foreign Application Priority Data
Jul. 12, 2002 (DE) 10231659.7

(51) Int. Cl. C12Q 1/68; C07H 21/04

(52) U.S. Cl. 435/6; 536/25.4

(57) ABSTRACT

Composition for bonding nucleic acid to a solid phase The present invention relates to a composition for bonding nucleic acid in aqueous solution to a solid phase containing a guanidinium salt, a buffer substance and a detergent, characterised in that the solution's pH value is ≧7.0.

The invention moreover relates to

a kit for isolating nucleic acid, containing the following component parts:

a) an aqueous nucleic acid-stabilising solution containing the following component

a guanidinium salt;

a buffer substance;

a reducing agent, and/or

a detergent;

b) a composition according to one of the claims 1 to 9; and

c) a solid phase capable of bonding nucleic acid; and

a method for isolating nucleic acid.

The present invention relates to a composition for optimising bonding of nucleic acid, preferably derived from blood and in an aqueous solution, to a solid phase as well as a kit for isolating nucleic acid.

In WO 00/09746 a vessel for taking blood is described which contains a solution comprising as its components a guanidinium salt, a buffer substance, a reducing agent and/or a detergent. The vessel is particularly suitable for taking blood that is to be examined for nucleic acids.

WO 01/60517 describes a vessel for taking samples containing a solution stabilising nucleic acid and a nucleic acid-bonding solid phase. The vessel is particularly suitable for taking blood that is to be examined for nucleic acid.

During taking of blood, for instance, the latter is conventionally collected in vessels which already contain anticoagulants such as heparin, citrate or EDTA. In this way, coagulation of the blood is prevented. Blood samples obtained in this way can be stored for longer periods of time at suitable temperatures. This method of taking blood has, however, significant disadvantages if the nucleic acids such as mRNA or viral RNA and DNA are to be analysed. For such purposes, the nucleic acids contained in the sample should preferably be stabilised at the moment of blood taking, that is, the degradation of nucleic acids present as well as re-synthesis of mRNA is to be prevented.

This goal of stable storage of the nucleic acids contained in the test material from the moment of blood taking has thus far been practically impossible, in particular when blood is stored, for the following reasons.

Cells contain nucleases, in other words enzymes which destroy nucleic acids as soon as they come into contact with their substrata (RNA, DNA). The impact of cellular and extra-cellular nucleases is normally under physiologic control as long as the cells are in their normal environment. Taking of blood entails more or less significant changes in the nucleic acids contained in the cells. Nucleases are then set free inside the cells and/or by means of the lysis of cells outside as well. In addition, nucleic acids are more or less strongly synthesised. Precisely long-term storage of biological samples, such as blood, entails ageing and destruction of the cells.

The problems of nucleic acid stability in blood samples described above also apply in a similar way to nucleic acids from other biological samples, such as samples of spittle and tissue.

A further problem in long-term storage of biological samples (such as blood) recovered with conventional test taking methods is the significant change of the test material. Such changes, such as strong lysis of cells, may entail the standard methods of nucleic acid isolation no longer functioning with satisfactory efficiency and replicability.

Apart from the problems of stable storage of nucleic acids contained in test material, further difficulties emerge from conventional methods of taking the samples (such as blood). For example, the conventional anticoagulants are frequently not separated with sufficient efficiency when isolating nucleic acid and interfere in subsequent nucleic acid analysis such as in cases of amplification by means of PCR (Polymerase Chain Reaction). Heparin, for instance, is a generally known inhibitor of PCR.

Finally, in quantitative nucleic acid analysis the question is raised how the entire method from taking of the sample up to measurement of the nucleic acid can be controlled and optimised under standardised conditions. Ideally, the test material should be fed a standard nucleic acid defined in quantity and quality already upon the taking of the sample and which is subjected to the entire process from taking of the sample up to determination. The nucleic acid originally contained in the sample should also, as far as possible, be fed quantitatively to the analysis. This is in particular of importance in diagnostics since in this context, depending on the findings, different consequences can emerge for treatment of the donor of the sample. This too cannot be accomplished with the conventional systems of sample taking and isolation.

A further disadvantage in conventional taking of samples, such as blood samples, is the danger of transferring infectious material since up until now manual process steps have been necessary for isolating nucleic acid. Contact with potentially infectious germs cannot be excluded.

In professional literature a method has been described in which a blood sample is mixed with guanidinium salt immediately after being taken from the patient (EP 0 818542 A1). With this method the guanidinium salt is present in the form of powder in order to take advantage of guanidinium salt's greater stability. However, this method has serious disadvantages since the salt, for instance, must first be dissolved in the blood added. The process of solution is, in particular, dependent upon the temperature and cannot be controlled due to the non-transparent test material used. The use of a corresponding product for diagnostic-medical purposes is thus extremely problematic.

Nucleases are extremely active enzymes occurring in high concentrations in particular in body fluids/secretions such as spittle or blood and which can only be inhibited under extremely denaturing conditions. Denaturing is dependent on the concentration of the guanidinium salt in solution. An inhibiting concentration of guanidinium salt in solution has not been present in the method in EP 0 818 542 from the very beginning. Therefore uncontrolled degradation of nucleic acids ensues during the solution process. With this method, addition of reducing agents is additionally dispensed with without which effective inhibition, in particular of RNases, is generally not ensured. Finally, EP 0 818 542 does not provide any measures for nearly quantitative isolation of the test material's nucleic acid.

The sample obtainable with conventional methods can furthermore not be directly used for additional nucleic acid isolation in solid phases. The use of guanidinium salt powder does not moreover allow for the addition of internal nucleic acid standards. However, such standards are indispensable for process control and precise quantification.

The object of the present invention is the technical problem of indicating means for optimising the yield of nucleic acids from biological samples, in particular to indicate means to optimise the bonding of nucleic acids from the sample to a solid phase. Finally, the means adopted should make it possible to use an enhanced nucleic acid analysis method for analysing nucleic acids from biological samples with a lower detection limit and in which case this is particularly desirable in the context of diagnostics.

This problem is solved by the invention by means of a composition for optimising the bonding of nucleic acid in aqueous solution to a solid phase (a bonding solution also known as Pr1S), containing a guanidinium salt, a buffer substance and a detergent, characterised in that the pH value of the solution is ≧7.0, preferably <7.5 and most preferably <8.0.

This problem is also solved with a kit for isolating the nucleic acid containing the following components:

a) an aqueous solution for stabilising nucleic acid (also known as N-sS or NAST), containing the following components:

a guanidinium salt, and/or

a buffer substance, and/or

a reducing agent, and/or

a detergent;

b) a composition for optimising bonding of nucleic acid in aqueous solution to a solid phase containing guanidinium salt, a buffer substance and a detergent, characterised in that the solution's pH value is ≧7.0, and

c) a solid phase which can bond nucleic acids.

Additional preferred embodiments are indicated in the subclaims.

The kit offers the following advantages: 1. The sample, preferably blood, goes through lysis immediately when it is taken in that the vessel for taking it already contains a corresponding lysis solution which is simultaneously a nucleic acid-stabilising solution. 2. The nucleic acid-stabilising solution entails the test material, in particular the nucleic acids contained therein, being stabilised immediately upon contact with the solution. 3. The nucleic acid-stabilising solution has moreover been chosen so that the test material can be directly used in the subsequent isolation processes. 4. The nucleic acid-stabilising solution can be separated out so efficiently in subsequent isolation that inhibition of, e.g. the PCR does not occur. 5. An internal standard can be added to the nucleic acid-stabilising solution. This internal standard allows for control of the entire process from taking of the sample up through detection of nucleic acid. 6. The solid phase contained in the vessel is particularly suitable for subsequent isolation of the nucleic acid bound to it. 7. The compositions's addition to bonding the nucleic acid to a solid phase, “bonding solution”, entails, without being bound to a specific theory, release of the nucleic acids from any eventually generated precipitates of blood components and enhanced bonding of the nucleic acid to the solid phase and thus to an increased yield of isolated nucleic acid available for analytic purposes. In addition, by means of the bonding of nucleic acid to the solid phase, subsequent isolation is simplified by having an initial separation of nucleic acid and additional test components occur in the vessel.

The nucleic acid-stabilising solution can be chosen such that the nucleic acid immediately after cell lysis bonds to the corresponding surface or only does so after additional reagents are added. The first case is, for instance, given if a glass surface is specified in the presence of a guanidinium salt. The second case can be attained or optimised by adding the “bonding solution” or, for instance, when a biotin-coated surface is provided with subsequent addition of streptavidin with nucleic acid-bonding properties.

The kit can basically be used for processing of any body fluids whatsoever and is particularly suited to processing body fluids containing cellular components such as bone marrow or, as an example, spittle samples. However it preferably implies a kit for direct taking of whole blood from a donor.

The kit preferably contains a vessel that preferably consists of a conventional vessel for taking blood (such as a tube) in which a defined volume of a nucleic acid-stabilising solution and a nucleic acid-bonding solid phase are contained. The tube is subsequently and preferably provided with a predefined low pressure making it possible for a specific volume of blood to be taken. The tube can be used with conventional methods for taking blood. The stabilising solution contained in the tube contains the following reagents in the preferred embodiment:

A guanidinium salt such as guanidinium thiocyanate, a detergent such as Triton-X-100, a reducing agent such as dithiothreitot and a suitable buffer system such as citrate, Tris, MES or HEPES. In the composition described, the solution is compatible with the vacuum tube. The solution can be stored in the vacuum tube without any problem and without any impairment of the stabilising function desired ensuing. The entire system is, in particular, safe and free of any problems for the donor when the sample is taken.

The nucleic acid-stabilising solution, containing the guanidinium salt serving as a lysis substance and stabilising substance, the solid phase bonding the nucleic acid, the buffer substance, the reducing agent and the detergent can be stored stable and convert the freshly taken material added, such as blood, into a material that is likewise stable when stored and which can be used directly for additional nucleic acid analysis or isolation.

As guanidinium salt guanidinium thiocyanate and/or guanidinium chloride are preferred.

Preferably the guanidinium salt should be available in a concentration of 1 to 8.0 M.

As buffer substance, Tris or citrate is preferred, in which case the exact pH is preferably fixed with HCl. Additional possible buffers are, however, HEPES, MOPS, MES, citrate and phosphate buffers like PBS.

Deployable as solid phases are all materials which bond nucleic acids. Particularly suitable are glass particles, polymers which bond nucleic acid, particles coated with the same, coatings of the system for taking blood bonding nucleic acid or particles coated with silica. The surface of the solid phase bonding the nucleic acid can by way of an alternative be coated with specific bonding molecules (such as streptavidin, oligo-nucleotides, peptide nucleic acids (PNA), etc) which interact directly with marker molecules on the nucleic acid or directly with the nucleic acid. The shaping of the materials is only dependent on the shape of the system for taking the samples and on the subsequent isolation method. Particularly suitable are shapes deployable directly subsequent to or during further processing of nucleic acid and especially suitable are surfaces compatible with conventional isolation methods such as magnetic particles or fleece.

Suitable solid phases are commercially available such as magnetic particles coated with silica as they are contained in the mRNA Isolation Kit for Blood/Bone Marrow (ROCHE).

The buffer concentration in the nucleic acid-stabilising solution should preferably lie in the range of 10 to 300 mM, particularly desirable being the range from 10 to 100 mM.

Triton-X-100 is preferred as detergent in the nucleic acid-stabilising solution. Other possible detergents are NP-40, Tween 20, Polydocanol or other detergents.

The detergent concentration in the nucleic acid-stabilising solution lies preferably in the range from 5 to 30% (w/v), particularly preferable being from 10 to 20% (w/v).

Preferred as reducing agent is DTT; however, also P-mercapto-ethanol, TCEP (Tris(2-carboxyethyl)phosphin) or other reducing agents can be deployed.

The preferred concentration of the reducing agent in the nucleic acid-stabilising solution lies from 0.1 through 10% (w/v) particularly preferred is the range from 0.5 through 2% (w/v).

The pH in the nucleic acid-stabilising solution lies preferably in the range from 2.0 to 9.0 and particularly preferred in that between 4.0 and 7.5.

The pH-value of the solution is selected in particular so that after addition of the test material a pH value in the range from 5.0 through 7.5 establishes itself in the nucleic acid-stabilising solution. Since by specifying a low pressure it is ensured which sample volume is taken, it can be ensured by specifying a desired buffer concentration or a corresponding volume of solution that after the entire test volume has been absorbed the desired pH will also be achieved. Particularly preferred is a pH between 6.3 and 6.9 after the sample has been taken.

A particularly preferred nucleic acid-stabilising solution contains some 3-4 M of guanidinium thiocyanate, 40-80 mM of Tris, 11-14% (w/v) of Triton-X-100, 40-80 mM of DTT, a solid phase of glass particles or silica-coated magnetic particles, in which case the pH is fixed so that after addition of blood a pH of between 6 and 7.5 results.

In another preferred embodiment the volume for absorption of the blood sample has a low pressure which can be set so that a predefined volume of blood is sucked into the vessel for taking the blood after a blood vessel has been pierced. Correspondingly evacuated vessels are available on the market.

The vessel containing the blood taken can then be immediately sent on to the next steps in analysis or else stored for a protracted period of time (up to several days or weeks) without adverse effects on the sample's quality.

With the method according to this invention the freshly taken sample, such as blood, is brought into contact directly in the vessel for taking the sample with the nucleic acid-stabilising solution described above so that immediately all processes which can alter the nucleic acid pattern in the sample are stopped. The nucleic acids can, in the vessel, preferably be present already bonded to the solid phase or can be bonded to the solid phase in a further reaction step, in which case the extent of bonding by means of addition of the bonding solution according to the present invention is optimised.

The data later computed in the context of nucleic, acid analysis in regard to the detected nucleic acids therefore constitute very precisely the actual condition at the time when blood is taken, both in regard to quantity as well as in regard to the types of nucleic acid.

The volume of blood taken corresponds preferably to 0.1-times to 2-times of the solution placed in the vessel, the latter amounting preferably to some 0.5 to 5.0 ml. The final concentration of guanidinium salt after addition of the sample thus lies in the range from 1.0 to 5.0 M, preferably 1.0 to 3.0 M, before the bonding solution is added.

After administration of the bonding solution into the vessel containing the test material, such as blood, and the nucleic acid-stabilising solution, the solution in the vessel will preferably contain:

a guanidinium salt in a concentration from 1 to 8 M;

a detergent in a concentration of some 5 to 25% (w/v);

a buffer in a concentration of some 100 to 500 mM;

a reducing agent in a concentration of some 5 to 50 mM, and will have a pH value of <7.5 and preferably of ≧8.0.

In a particularly preferred embodiment, the vessel with the blood sample, stabilising solution and bonding solution will contain the following components:

a guanidinium salt in a concentration from 1.5 to 5, preferably from 2.5 to 3.5 M;

a detergent in a concentration from 8 to 20, preferably from 10 to 16% (w/v),

a buffer in a concentration from 150 to 400, preferably 200 to 300 mM)

a reducing agent in a concentration from 20 to 40 mM, preferably from 25 to 35 mM; and with

a pH <7.0, preferably <7.5 and, particularly preferred, ≧pH 8.0.

It is moreover preferred that the solution cited above from the blood sample, NsS and Pr1S possesses a pH≦10, preferably ≦pH 9.0. This measure minimises any alkaline hydrolysis of the nucleic acid.

The kit according to the invention is preferably deployed for taking the sample if the test sample is to be used for analysing nucleic acid.

The use of the nucleic acid-stabilising solution cited above as a component part of the sample-taking system described guarantees immediate lysis of the cells and simultaneous stabilisation of the sample by means of direct inactivation of the nucleases. Surprisingly enough, the sample thus obtained can be stored for several days even at room temperature. The sample-taking system furthermore ensures handling which is non-infectious and safe from contamination from the sample-taking and isolation of the nucleic acid up through analysis. With conventional methods of nucleic acid isolation, up until now additional handling steps (such as transferring the blood taken into the reagents for isolating the nucleic acid, etc) have been necessary which have been linked to an additional risk of infection or contamination of the sample, as described in detail in the introduction. Although the kit has essentially been described in connection with a vessel for taking blood, what has been said also applies to other systems for taking biological samples such as swabs.

Surprisingly enough, the nucleic acid partially bonded to the solid phase can be isolated from the test material simply, even after protracted storage. During storage of the stabilised nucleic acid, with increasing storage duration increasingly precipitates can be generated consisting of blood components such as porphyrin salts of haemoglobin to which nucleic acid to some extent bonds. The presence of the bonding solid phase during sample lysis and stabilisation entails immediate bonding of some nucleic acids, primarily DNA, to the surface. Only when bonding solution is added is a complete release of the nucleic acid from any eventually generated precipitates and their optimum bonding to the solid phase is achieved. The addition should occur immediately prior to the actual isolation step since due to administration of the bonding solution optimum stabilisation of the nucleic acid can no longer be guaranteed. Addition of the bonding solution occurs preferably immediately prior to actual processing of the sample for isolation of the nucleic acid.

The sample recovered with the kit can be used with customary nucleic acid isolation methods, when silica-coated magnetic particles or silica-fleece in columns are used it is possible to fall back on customary standard methods of nucleic acid isolation (magnetic separation or centrifugation or by subjecting the nucleic acid to low pressure or washing or eluting it).

The present invention thus consists of a system for taking samples designed in such a way that the following conditions are met: 1. Controlled sample taking and simultaneous stabilisation of the nucleic acids (DNA, RNA) contained in the test material. 2. Sample taking where the use of anticoagulants can be entirely dispensed with. 3. Optimised bonding of nucleic acids to a solid phase contained in the system. 4. The sample recovered with the system described can be easily integrated into existing nucleic acid isolation systems. 5. The system, including the sample contained in it, is stable when stored.

It was additionally and surprisingly discovered that the sample recovered with the sample-taking system described is stable when stored in the vessel for a protracted period of time without any degradation of the nucleic acids.

The following examples illustrate the invention.

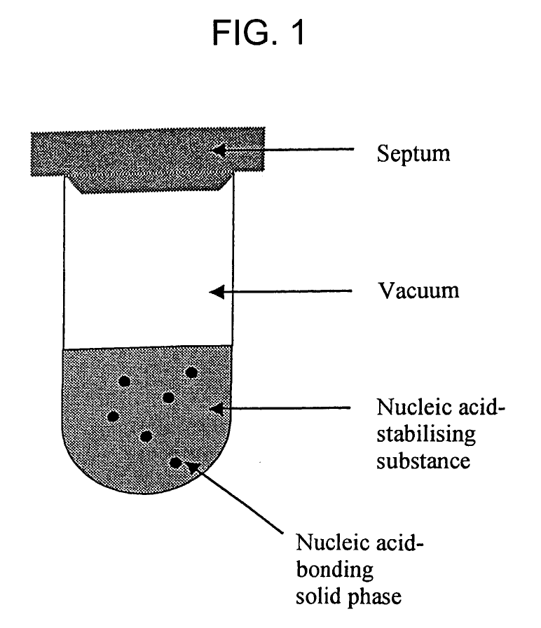

FIG. 1:

Vessel for taking samples with nucleic acid-stabilising substance (N-sS), predefined vacuum, laced with solid phase and sealed with septum.

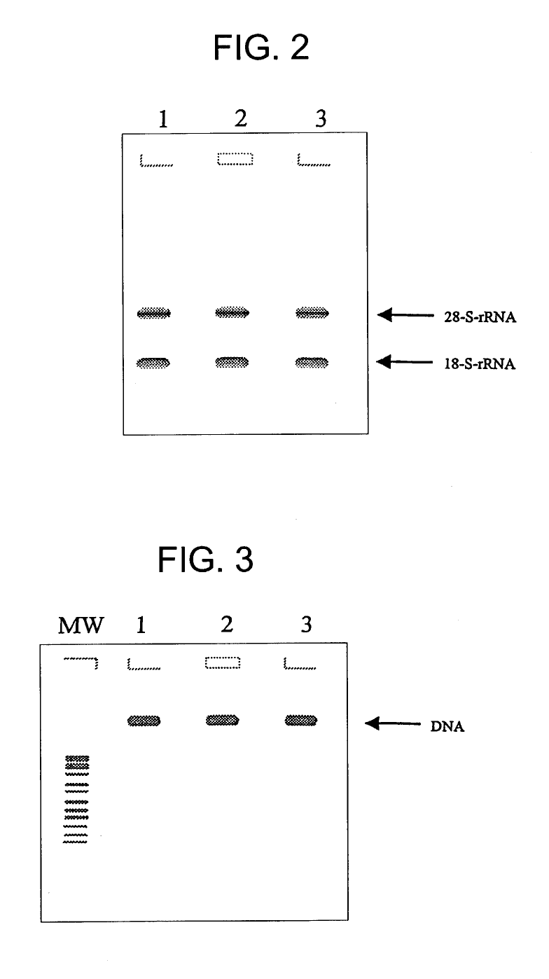

FIG. 2:

Graphic representation of a gel analysis (1% agarose) of 28S and 18S rRNA stored for varying periods of time in the sample-taking vessel.

Column 1: isolation and fractionation of RNA immediately after the sample is taken (no storage); Column 2: storage for one month at −20° C. Column 3: storage for six days at 4° C. The quantity of the RNA applied corresponds to a blood volume of 120 μl.

FIG. 3:

Graphic representation of a gel analysis (1% agarose) of DNA stored for varying periods of time in the sample-taking vessel.

Column 1: isolation immediately after the sample is taken (no storage); Column 2: storage for one month at −20° C. Column 3: storage for six days at 4° C. The quantity of the DNA applied corresponds to a blood volume of 10 μl.

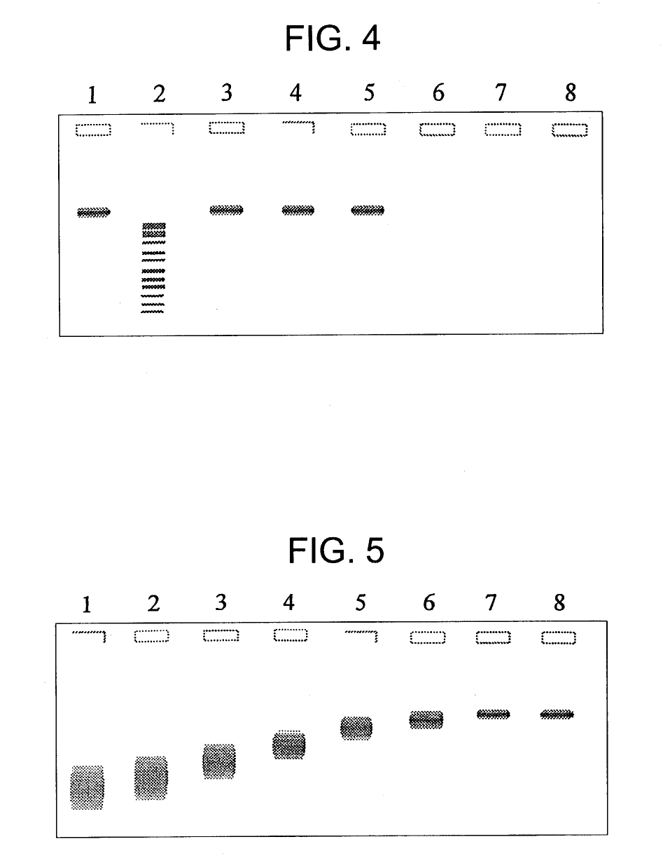

FIG. 4:

Graphic representation of a gel analysis of isolated MS2-RNA after incubation in serum/stabilising solution with/without DTT after 180 minutes at 40° C.

Column 1: positive control: MS-2 RNA; Column 2: DNA marker; Columns 3 through 5:MS-2 RNA after incubation with stabilising solution containing DTT (triple determination); Columns 6 through 8: MS-2 RNA after incubation with stabilising solution without DTT (triple determination).

FIG. 5:

Graphic representation of a gel analysis of MS2-RNA isolated after incubation in serum/stabilising solution for three days at 40° C. The guanidinium thiocyanate (GTC) content of the stabilising solution after addition of the serum, in which the relevant RNA was incubated, is indicated in the relevant column.

Column 1: 2.70 M GTC Column 2: 2.5 M GTC; Column 3: 2.36 M GTC Column 4: 2.2 M GTC Column 5: 2.08 M GTC, Column 6: 1.94 M GTC; Column 7: 1.80 M GTC1 Column 8: 1.66 M GTC.

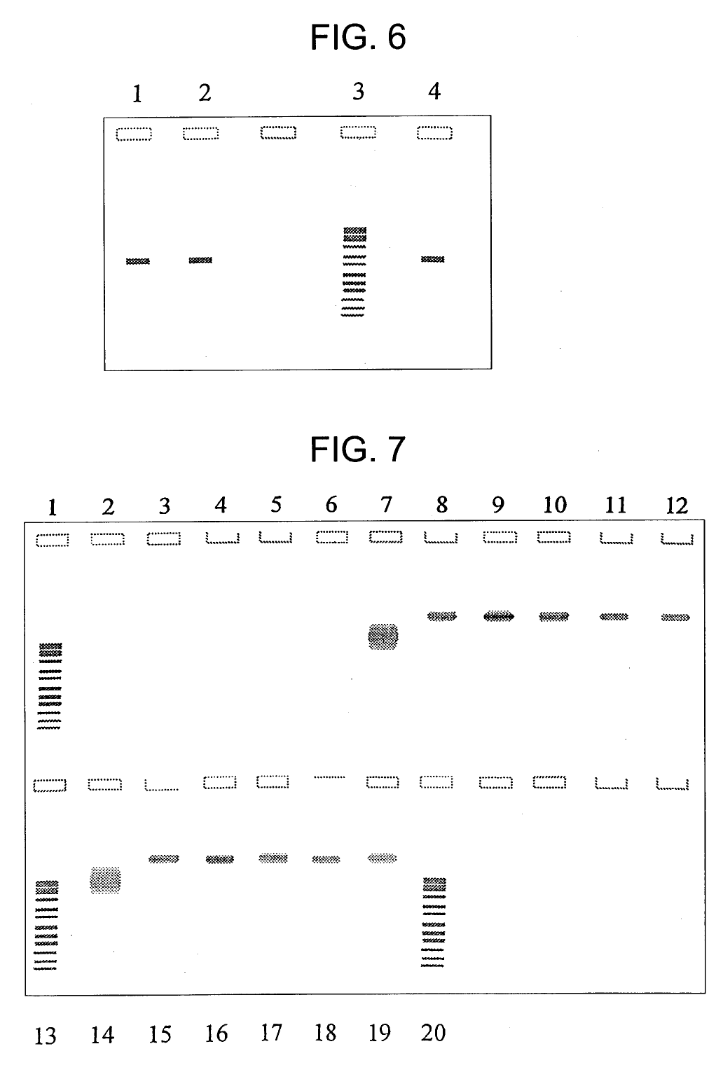

FIG. 6:

Graphic representation of a gel analysis of the PCR amplification products of MS2-RNA isolated after one or eight days of incubation at 40° C. in serum/stabilising solution.

Column 1: amplification product of RNA isolated after one day; Column 2: amplification product of RNA isolated after eight days; Column 3: DNA marker; Column 4: MS2-RNA positive control. 0.8 μg in 10 μl RT 1:50 diluted, 1 μl amplified.

FIG. 7:

Graphic representation of a gel analysis of isolated MS2-RNA after six (Columns 2-12) or 13 (Columns 14-19) days of incubation at room temperature in serum/stabilising solution. Behind the relevant columns the pH value is indicated which was achieved after mixing of serum and stabilising solution.

Columns 1, 13, 20: DNA marker; Column 2: pH 8.0; Column 3: pH 7.7; Column 4: pH 7.5; Column 5: pH 7.35; Column 6: pH 7.18; Columns 7, 14: pH 7.07; Columns 8, 15: pH 6.94; Columns 9, 16: pH 6.8; Columns 10, 17: pH 6.72; Columns 11, 18: pH 6.68; Columns 12, 19: pH 6.7. The stabilising solution of RNA in Columns 12, 19 had the same pH value as the RNA in Column 11, but contained 5 M 4 M GTC instead of 4 M 3 M.

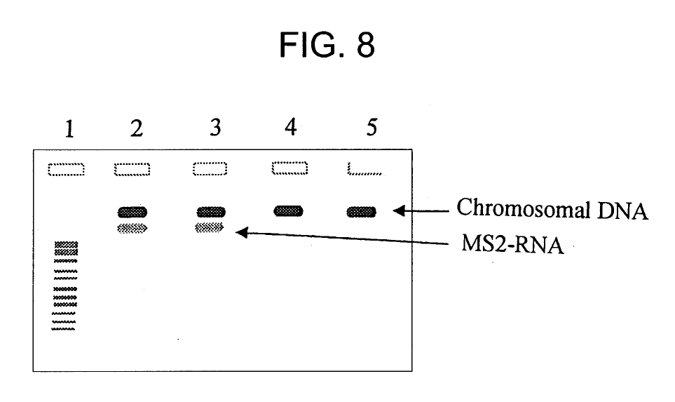

FIG. 8:

Graphic representation of evidence of RNA and DNA in standard agarose gel (1% agarose). Column 1: molecular weight marker; Columns 2 through 4: isolated nucleic acids Column 2: nucleic acid from whole blood lysate laced with MS2-RNA (seven days); Column 3: nucleic acid from whole blood lysate laced with MS2-RNA (zero days, control); Column 4: nucleic acid from whole blood lysate (seven days); Column 5: nucleic acid from whole blood lysate (zero days, control). The upper bands show chromosomal DNA (clearly recognisable in all four samples), the lower bands in Columns 2 and 3 show the added and isolated MS2-RNA.

### EXAMPLE 1

### Blood-Taking System

In a preferred embodiment the blood-taking system can consist of the following structure (see FIG. 1): A tube is filled with a predefined volume of nucleic acid-stabilising solution, provided with a nucleic acid-bonding solid phase and with a predefined vacuum and then closed with a septum. The septum is designed so that it is compatible with conventional sample-taking accessories (cannula, etc). In the present example 2.2 ml of reagent were provided and the vacuum was adjusted so that when a sample is taken exactly 1.3 ml of blood are able to flow in. The nucleic acids contained in the blood flowing in were immediately transferred to a stable form.

General preliminary remark on the subsequent examples:

Unless otherwise mentioned, in all of the examples described here below, the nucleic acid-stabilising substance (N-sS) had the following composition: 45 mM of Tris, 5 M of guanidinium thiocyanate, 0.8 (w/v) dithiothreitol, 18% (w/v) Triton-X-100, pH 6.0.

In all the examples described, the nucleic acid-stabilising substance was mixed with the sample in the ratio of 1 to 0.59 (I volume of N-sS plus 0.59 volume of test material).

For all examples blood was stabilised by having it put in the tube laced with N-sS immediately after being taken.

### EXAMPLE 2

### Stability of Nucleic Acid after Mixing the Test Material and N-sS

### Isolation of RNA and DNA from the Test Lysate with Silica-Derivative Surfaces

### Materials and Method

The test material for DNA and RNA isolation was used immediately after being taken, after storage for six days at 4° C. and after storage for one month at −20° C.

For isolation of RNA (FIG. 2) the High Pure RNA Isolation Kit (ROCHE, cat no 1 828 665) was used. The instruction leaflet regulation was modified in the following manner. A volume of 2.4 ml of test lysate was applied in four aliquot parts with 600 μl each to the column so that a total of test material was applied from 2.4 ml of lysate. All other steps were carried out as per the instruction leaflet. The RNA was finally eluted with 100 μl of elution buffer.

To isolate DNA (FIG. 3) the QiaAmp Blood Kit (QIAGEN, cat no 29104) was deployed. The standard procedure described in the instruction leaflet was modified in different points. 400 μl of test volume were placed directly on the column in which context the bonding reagent contained in the kit was not used. 25 μl of proteinase K stick solution were added and the sample incubated for ten minutes at room temperature. Thereafter, the column was placed in a collector vessel and centrifuged as described in the instruction leaflet. All further steps, with the exception of the use of ethanol, were carried out as described in the instruction leaflet. The elution volume was 200 μl.

### EXAMPLE 3

### Significance of Reducing Reagents (e.g. DTT) in the Stabilising Solution for Long-Term Stabilisation of RNA

### Materials and Method

Stabilising solution used:

4.0 M GTC; 13.5% of Triton-X-100; 45 mM of Tris/HCl; with 120 mM DTT or without DTT. 700 μl of serum were mixed with 700 μl of stabilising solution. After two minutes of incubation, 20 μl of MS2-RNA (0.8 μg/μl from ROCHE Diagnostics) were added. The samples were incubated for 180 minutes at 40° C. and subsequently processed in aliquot parts of 400 μl with the High Pure Total RNA Kit from ROCHE in accordance with Experiment 1. The samples were eluted in 50 μl and frozen at −20° C. Analysis occurred by means of agarose gel (see FIG. 4).

### Result

Without the addition of reducing reagents to the stabilising solution, long-term stabilisation of RNA cannot be achieved.

### EXAMPLE 4

### Stability of MS2-RNA in Serum/Stabilising Solution: Dependence on GTC Concentration

### Materials and Method

Stabilising solutions used: 3-5 M GTC; 13.5% Triton-X-100; 50 mM of DTT; 42 mM of Tris/HCl;

pH of the solutions: about 5.0;

pH of the solutions after addition of serum: about 6.7.

2.0 ml of serum were mixed with 2.5 ml of each of the stabilising solutions. After an incubation period of 2-5 minutes, 90 μl of MS2-RNA (0.8 μg/μl from ROCHE) were added and incubated at 40° C. At regular intervals, 400 μl samples were taken and processed with the High Pure Total RNA Kit from ROCHE in accordance with Experiment 1. The samples were eluted in 50 μl and frozen at −20° C. For analysis of RNA integrity, 20 μl of the elution product were applied to a 1.5% agarose gel (FIG. 5). The RT-PCR analysis was accomplished by means of AMV-RT and PCR. In each case, 10 μl of elution product were reverse transcribed by means of AMV-RT (ROCHE) and subsequently analysed on the Light Cycler by means of quantitative PCR.

| | |
|---|---|
| Preparation for RT: | 4.0 μl AMV-RT buffer |
| (42° C. for 1 hour) | 2.0 μl dNTPs (end concentration 10 mM) |
| | 0.5 μl RNase inhibitor (ROCHE, 20 units) |
| | 1.0 μl Primer 2827 (end concentration 1 μM) |
| | 1.9 μl DMPC water |
| | 0.6 μl AMV-RT (ROCHE, 15 units) |
| | 10 μl Template RNA |
| | 20 μl |

The PCR was carried out on the Light Cycler at an annealing temperature of 61° C. with the use of SYBR-Green as a detection system. All samples with a threshold cycle greater than 20 were considered negative since the signal detected is exclusively due to the formation of primer dimers. This can be conclusively proven by means of analysis of the melting graphs on the Light Cycler (ROCHE). The RT product was diluted 1:50 with bi-distilled water and 1 μl of it was used for a 10 μl PCR according to the following scheme:

| | |
|---|---|
| Preparation for PCR: | 1.6 μl MgCl₂ (parent solution, 25 mM) |
| | 5.9 μl DMPC water |
| | 0.25 μl Primer 2827 (parent solution, 20 mM) |
| | 0.25 μl Primer 2335 (parent solution, 20 mM) |
| | 1.0 μl SYBR-Green-Mastermix (ROCHE) |
| | 1.0 μl RT preparation |
| | 10 μl |

The amplified product of PCR was completely applied to a 2% agarose gel (see FIG. 6).

### Result

FIG. 5 shows the eluted MS2-RNA after three days of incubation at 40° C. as detected in agarose gel. Although after eight days at 40° C. all RNA samples can be amplified and unequivocally be detected (FIG. 6), after only three days clear differences can be seen in RNA integrity as a function of the GTC content. Accordingly, a salt content less than 2 M in the serum/stabilising solution is an advantage for RNA integrity, in particular at higher temperatures such as 40 degrees Celsius.

What is not shown is the fact that MS2-RNA as early as two minutes after being added to the serum is completely broken down by RNases and that no more RNA can then be shown to be detected. With this example it was possible to prove that the degradation of RNA by the addition of stabilising solution to the serum can be significantly retarded. After eight days at 40° C. in serum/stabilising solution MS2-RNA can be detected without any problems by means of PCR (FIG. 6), although RNA integrity suffered to some extent.

### EXAMPLE 5

### Stability of MS2-RNA in Serum/Stabilising Solution: Dependence on the pH Value of the Sample Laced with Stabilising Solution

### Materials and Method

| | | |
|---|---|---|
| Solution used: | 4 M (5 M) | GTC |
| | 14.4% | Triton-X-100 |
| | 50 mM | DTT |
| | 45 mM | Tris HCl |

pH after addition of serum between 6.7 and 7.5.

2.5 ml of stabilising solution were mixed with 2.0 ml of serum. After addition of 90 μl of MS2-RNA (0.8 μg/ml, ROCHE) the samples were incubated at room temperature. At regular intervals the RNA from a 500 μl sample was processed in accordance with Example 4 with the ROCHE Viral RNA Kit and isolated in 50 μl of elution buffer. 20 μl of the elution product were analysed with the aid of agarose gel (see FIG. 7).

### Result

The pH of the serum/stabilising solution and thus as well the pH and buffer range of the stabilising solution are crucial for long-term stabilisation of RNA. While at a pH value of 8.0 after only two days no intact RNA can any longer be demonstrated, in a pH range between 6.6 and 7.0 intact RNA can still be demonstrated after 13 days of incubation at room temperature. Apart from the pH value, however, an optimally adjusted GTC concentration is also of significance for long-term stabilisation of RNA (see Example 4). The example presented makes it clear that for any long-term stabilisation of RNA a GTC end concentration in the stabilised sample of 2.2 M GTC is better than 2.8 M.

### EXAMPLE 6

### Stability of a Nucleic Acid-Bonding Surface in the Presence of Stabilising Solution Shown by Using Magnetic Particles Coated with Silica

### Materials and Method

| | | |
|---|---|---|
| Solution used: | 4.5 M | GTC |
| | 15% | Triton-X-100 |
| | 100 mM | DTT |
| | 50 mM | MES |

In doing so, the solution and blood are deployed in a ratio of 1:1.

The silica-coated magnetic particles were taken from the mRNA Isolation Kit for Blood/Bone Marrow (ROCHE Molecular Biochemicals). The quantity of particles used per ml came to about 35 mg. The system for taking blood, consisting of a sample-taking tube, the stabilising solution and the magnetic particles, was stored for fourteen days at room temperature. Subsequently, whole blood was taken with the same system. As a control a freshly produced system for sample-taking (tube, stabilising solution, magnetic particles) was used. From both preparations, isolation of the nucleic acids contained in the test materials was accomplished. The magnetic particles were separated by means of a magnet, the overage being discarded. The particles were re-suspended in 50% ethanol, 10 mM of Tris, pH 7.0, and washed repeatedly with the same solution. Finally, the particles were heated in 10 mM of Tris/HCl (pH 7.0) up to 70° C., in the process of which the nucleic acid separated from the magnetic particles. The particles were separated magnetically and the overage containing nucleic acid was analysed in the standard agarose get.

### Result

TABLE 1

| | Sample | Control |
|---|---|---|
| | (14 days, RT) | (0 days) |
| Nucleic acid detectable | + | + |
| in the gel | | |

After 14 days of storage the solid phase's property of being able to bond nucleic acid was unchanged. The sample as well as the control show the same properties capable of bonding nucleic acid.

### EXAMPLE 7

### Stability, Isolation and Demonstration of DNA and RNA after Seven Days of Storage with Simultaneous Bonding to Silica-Coated Magnetic Particles

### Materials and Method

| | | |
|---|---|---|
| Suspension used: | 4.5 M | GTC |
| | 15% | Triton-X-100 |
| | 100 mM | DTT |
| | 50 mM | MES |
| | 35 mg/ml | Particles |

Four blood-taking systems (tubes) containing 1.0 ml of the suspension described above were laced with 1 ml of whole blood. Two of the tubes (whole blood lysate) were additionally laced with 25 μg MS2 of RNA. Each tube of the two preparations (whole blood lysate +/−MS2-RNA) was immediately thereafter used for nucleic acid isolation (for procedure, see Example 6). The two other tubes were stored for seven days at room temperature. After this period of time, isolation of the nucleic acid was carried out. The elution volume came to 200 μl per 200 μl of the whole blood volume. The nucleic acids were analysed in the standard agarose gel.

### Result

After seven days of storage in the sample-taking system (solution, solid phase) the stability of chromosomal DNA and MS2-RNA was demonstrably present (FIG. 8).

### EXAMPLE 8

### Extraction of mRNA as well as Cellular and Viral DNA by Bonding to Magnetic Polymer Beads on the Basis of Polyvinyl Alcohol and to Magnetic Silica Beads

| | |
|---|---|
| Test material: | 1.2 ml of stabilised blood (=400 μl of blood + 800 μl of NsS) |
| | (NsS = nucleic acid stabilising solution) |
| Spikes with viruses: | +6 × 10⁶ copies/ml of Cytomegaloviruses (CMV) |
| Nucleic acid extract: | +800 μl of bonding solution (PrlS = pre-incubation solution) |
| Magnetic beads: | a) 120 μl bead suspension of MagNA Pure LC Total Nucleic Acid |
| | Isolation Kit (cat no 3 038 505, ROCHE Molecular Biochemicals) |
| | b) 30 μl bead suspension of carboxyl-polyvinyl alcohol magnetic |
| | beads (M-PVA C 12, cat no 01-01.204) from the firm of Chemagen |
| | Biopolymer-Technologie AG, Baesweiler, GER |

Nucleic acid extraction protocol for a) and b):

Mix 900 μl of NsS-blood-Pr1S+a) 120 μl or +b) 30 μl of bead suspension

About fives minutes of incubation at room temperature

Magnetic separation

Remove overage completely

Proteinase K step:

2 mg PK/ml in 10 mM of TRIS-HCl, pH 6.5 with 0.1% Tween 20 and 0.5% Triton-X-100

Mix 500 μl of PK buffer per sample, 10 minutes of RT incubation

Magnetic separation, remove overage

First washing step:

Add 500 μl of washing buffer I (containing GTC) from the High Pure Viral Nucleic Acid Isolation Kit (cat no 1 858 874, ROCHE Molecular Biochemicals) per sample and mix for 10 seconds manually or with Vortex

Magnetic separation, remove overage completely

Second washing step:

Repeat same washing step with 500 μl of washing buffer I

Third washing step:

Add 900 μl of washing buffer II (containing ethanol) from the same kit as above per sample and mix

Magnetic separation, remove overage completely

Incubate the elution in 100 μl of elution buffer from the same kit as above at 80° C. for ten minutes and completely remove the elution product after magnetic separation and freeze at −70° C. until analysis

Analysis of nucleic acid:

Agarose gel analysis:

20 μl of the elution product are analysed on a 1% native agarose gel

Result for a) and b):

genomic DNA is visible

a) 100%

b) 80%

rRNA is visible

a) 100%, equivalent to about 10-20%

b) 200% of all cellular RNA

PCR for genomic DNA as exemplified by the G6P-DH gene:

Result:

a) 100%

b) 75%

PCR for CMV:

Result:

a) 100% (equivalent to 4×106 copies/ml=about 70% of the spiked CMV quantity)

b) 75% (equivalent to 3×106 copies/ml=50% of the spiked CMV quantity)

RT-PCR for G6P-DH mRNA:

Result:

a) Cannot be demonstrated, presumably because the GTC content for bonding of mRNA was too low due to dilution with the proteinase K buffer

b) 50% in comparison to standard experiments with silica magnetic beads corresponding to MagNA Pure Total NA Isolation Kit from ROCHE Molecular Biochemicals, as used in this experiment

For assessing the results in the individual experiments, in each case the amount of nucleic acid detected with the magnetic beads with silica surface from ROCHE Molecular Biochemicals (=a) was rated as the standard and thus set at 100% and the quantity isolated with b) was set in relation to it.

Pr1S composition (bonding solution) in the experiment above:

3.5 M GTC; 10% of Triton-X-100; 350 mM of Tris/HCl; pH 8.0.

NsS composition (stabilising solution) in the experiment above:

3.5 M GTC; 12.5% of Triton-X-100; 60 mM of Tris/HCl; 60 mM of DTT.

### EXAMPLE 9

### Demonstration of Bonding Efficiency of Nucleic Acid to Silica Surfaces by Addition of Bonding Solution “Pr1S” to the NAST Blood Mixture

| | |
|---|---|
| PrlS composition: | 3.5 M GTC; 10% of Triton-X-100; 350 mM of Tris/HCl; pH 8.0. |
| NAST composition: | 3.5 M GTC; 12.5% of Triton-X-100; 60 mM of Tris/HCl; 60 mM of DTT. |
| Test material: | 580 μl of stabilised blood (200 μl of blood + 380 μl of NAST = |
| | ratio of 1:1.9) |
| Spikes with CMV: | 6 × 10⁶ copies/ml of Cytomegalovirus (CMV) |
| Nucleic acid extraction: | a) Addition of 580 μl of PrlS (=ratio of 1:1) |
| | b) Without addition of PrlS |
| Extraction protocol: | All necessary reagents such as magnetic beads with a silica |
| | surface, washing buffers and elution buffers from the MagNA Pure |
| | Total NA Isolation Kit ® from ROCHE Molecular Biochemicals |
| | (cat no 3 038 505) were used. The proteinase K is likewise from |
| | ROCHE Molecular Biochemicals with the cat no 1 964 364. |

a)

+40 μl

+300 μl

b)

+40 μl of proteinase K (20 mg/ml)

+300 μl of magnetic bead suspension

Mix and incubate for I10 minutes at room temperature

Magnetic separation and remove overage

First washing with 850 μl of washing buffer I

Magnetic separation and remove overage

Second washing with 450 μl of washing buffer II

Magnetic separation and remove overage

Third washing with 450 μl of washing buffer III

Magnetic separation and remove overage completely

Elution with 200 μl of elution buffer at 70° C. with 10 minutes of incubation

Magnetic separation and carefully remove overage=elution product and freeze at −70° C. until analysis

Analysis of the extracted nucleic acids:

Analysis of agarose gel:

20 μl of the elution product were fractionated on a native 1% agarose gel

Chromosomal DNA:

a) 100%

b) about 14%

rRNA:

a) 100%

b) about 20%

Quantitative determination of CMV with the Light Cyclerg®, ROCHE Molecular Biochemicals

a) 4.3×10⁶ copies/ml=72% of extraction efficiency

b) 1.0×10⁶ copies/ml=17% of extraction efficiency

Quantitative determination of the genomic DNA with the G6P-DH Gene:

a) 100%

b) 21%

Quantitative mRNA determination on the basis of G6P-DH mRNA:

a) 100%

b) 7%

All results of analyses carried out demonstrate that the addition of the “Pr1S” bonding solution is necessary for optimum extraction of the nucleic acids due to their optimum bonding to the silica solid phase.

### EXAMPLE 10

### Nucleic Acid Extraction from NAST Blood with Pr1S on Silica Surfaces with the High Pure Viral Nucleic Acid Kit® from ROCHE Molecular Biochemicals, Cat No 1 858 874

Taking blood: The taking of blood is accomplished in a NAST Vacuette ® tube from the
firm of Greiner BIO-ONE. This vacuum sample tube for taking blood has a total volume of 5 ml
and contains 2.3 ml of NsS (nucleic acid stabilisation) solution. The vacuum is adjusted so that
when blood is taken 1.5 to 1.25 ml of blood flow into the tube and mix in with the solution. In
this way, 3.5 ml of NAS blood mixture are present in the tube, and where the blood is diluted
1:2.8.
NsS = nucleic acid stabilising solution: 3 M GTC; 12.5% of Triton-X-100; 30 mM of MES; 120
mM of DDT.
PrlS = bonding solution: 4 M GTC; 12.5% Triton-X-100; 250 mM of Tris/HCl; pH 8.0.
200 μl of blood is the equivalent of 560 μl of blood NAS mixture.

| | |
|---|---|
| Spikes: | Each tube was spiked with a positive HCV and CMV plasma so that it had the |
| | following concentrations: |
| | HCV: 5.7 × 10⁵ IU/ml of blood NAS mixture |
| | CMV: 2.0 × 10⁶ copies/ml of blood NAS mixture |
| Storage: | The blood-taking tubes are stored for one day at 20-24° C. |

| | |
|---|---|
| Nucleic acid extraction: | Mix 560 μl of blood NsS mixture (=200 μl of blood) with 350 μl |
| | of PRIS and incubate for up to 15 minutes at RT with repeated |
| | mixing (Vortex) to dissolve the crystals. |
| | Add 115 μl of isopropanol, mix, apply in two portions to the High |
| | Pure ® column centrifuged in each case at 6000-7000 rpm for one |
| | minute. |
| | Apply 450 μl of Removal Washing Buffer ® (ROCHE kit |
| | component) and centrifuge at 6000-7000 rpm for one minute. |
| | Wash the column twice with 450 μl of washing buffer ® (ROCHE |
| | kit component), centrifuge each at 6000-7000 rpm, apply 100 μl of |
| | 70° C. hot elution buffer ® (kit component = water) for elution and |
| | centrifuge at 10,000 rpm for two minutes. |
| | Nucleic acids from 200 μl of blood are present in 100 μl of elution |
| | product and are stored until analysis at −70° C. in 10 μl aliquots. |

### Analysis

All steps in analysis were carried out in comparison with a standard nucleic acid extraction with the PAXgene Blood RNA Kit® from the firm of Preanalytix GmbH, Switzerland. This kit also works with whole blood and contains another system of nucleic acid stabilisation during the taking of blood.

1. Qualitative DNA/RNA analysis with agarose gel analysis

NAST-PRIS: The extracted nucleic acids consist to 90-95% of cellular RNA (ribosomal RNA, mRNA) while the chromosomal DNA lies as a thin bandwidth in the range from approx. 5 to 10%.

PAXgene: The extracted nucleic acids consist to some 20-70% of cellular RNA=rRNA and to some 30-80% of chromosomal DNA.

2. Quantitative DNA/RNA determination by means of photometric measurement (A 260)

| | |
|---|---|
| NsS-PRIS: | The 100 μl elution product (=0.2 ml of blood) contain 2 μg of RNA/DNA |
| | equivalent to 6.25% of all cellular nucleic acids. With a DNA:RNA split |
| | according to agarose gel of about 1:10 this is the equivalent of about 0.2 |
| | μg of DNA and 1.8 μg of RNA. |
| | This thus corresponds to a yield of about 1% of the genomic DNA and |
| | about 90% of the entire cellular RNA = rRNA. |
| PAXgene: | The 80 μl of elution product (=2.5 ml of blood) contain 8.7 μg of |
| | RNA/DNA, the equivalent of 0.7 μg from 0.2 ml of blood, thus giving a |
| | yield of 2% of all nucleic acids in the blood. In accordance with |
| | DNA:RNA = 30-80% : 70 − 20% split, fluctuating strongly from |
| | experiment to experiment, this is the equivalent of a yield of 0.7-1.8% of |
| | genomic DNA and of only 7-24% of all cellular RNA = inclusive rRNA. |

This means that with the NAST-PRIS according to this invention about three times more total nucleic acids and 4-13 times more total cellular RNA, largely consisting of rRNA, are isolated.

3. Quantitative HCV determination with the Light Cycler from the firm of ROCHE Diagnostics

| | |
|---|---|
| NsS-PRIS: | An HCV concentration of about 4 × 10⁵ IU/ml was measured. Set in |
| | relation to the spiked concentration of 5.7 × 10⁵ IU/ml this is the |
| | equivalent of 70% recovery. |
| PAXgene: | HCV concentrations of 0.6 to 1.0 × 10⁴ IU/ml were measured, |
| | corresponding to a recovery of 1-1.75% of the spiked 5.7 × 10⁵ of HCV |
| | IU/ml. |

Thus with the PAXgene system some 40-70 times lower yield is attained for HCV in comparison with the NAST-PRIS system.

4. Quantitative CMV determination with the Light Cycler from the firm of ROCHE Diagnostics

| | |
|---|---|
| NAST-PRIS: | A CMV concentration of about 8 × 10⁵ copies/ml was measured, being the |
| | equivalent of 40% recovery of the spiked concentration of 2 × 10⁶ |
| | copies/ml. |
| PAXgene: | CMV concentrations of 0.8 to 1.6 × 10⁵ copies/ml were measured, |
| | constituting the equivalent of recovery of 4-8% of the spiked 2 × 10⁶ of |
| | CMV copies/ml. |

In this way with the PAXgene system 5 to 10 times lower yields are attained in comparison with the NAST-PRIS system.

5. Quantitative mRNA (G6P-DH) determination with the Light Cycler from the firm of ROCHE Diagnostics

| | |
|---|---|
| NAST-PRIS: | 8-12 ng of G6P-DH mRNA were measured in 200 μl of blood. |
| PAXgene: | 0.1-0.25 ng of G6P-DH mRNA were measured in 200 μl of blood. |

With the NAST-PRIS system according to the invention 50 to 100 times more mRNA is isolated.

6. Quantitative determination of the G6P-DH gene with the Light Cycler from the firm of ROCHE Diagnostics

| | |
|---|---|
| NAST-PRIS: | 190-430 ng of G6P-DH genes were measured in 200 μl of blood. |
| PAXgene: | 544-1200 ng of G6P-DH genes were measured in 200 μl of blood. |

The experiments shown above demonstrate unequivocally that the system according to the invention entails a clear increase in the yield of low molecular nucleic acids, in particular of RNA (such as mRNA and viral RNA and DNA (e.g. HCV, CMV)). The concepts of NAST and NsS were used synonymously.

1. Composition for bonding of nucleic acids in aqueous solution to a solid phase containing a guanidinium salt, a buffer substance and a detergent, characterised in that the pH value of the solution is ≧7.0, preferably >7.5 and most preferably >8.0.

2. Composition according to claim 1 where the concentration of the buffer substance in the aqueous solution is at least 100 mM.

3. Composition according to claim 2 where the concentration of the buffer substance in the aqueous solution lies between 250 mM and 750 mM.

4. Composition according to claim 3 where the concentration of the buffer substance in the aqueous solution lies between 450 mM and 550 mM.

5. Composition according to one of the claims 1 to 4, characterised in that the guanidinium salt has been selected from guanidinium thiocyanate and guanidinium chloride.

6. Composition according to one of the claims 1 or 5, characterised in that the guanidinium salt is present in a concentration of from 1 M to 8 M.

7. Composition according to one of the claims 1 to 6, characterised in that the detergent has been selected from Triton-X-100, NP-40, Polydocanol and Tween 20.

8. Composition according to one of the claims 1 to 7, characterised in that the detergent is present in a concentration of from 5% (weight) to 30% (weight).

9. Composition according to one of the claims 1 to 8, characterised in that the aqueous solution contains the following component parts:
approximately 3-5 M of guanidinium thiocyanate;
approximately 12- 18% (w/v) of Triton-X-100;
approximately 450-550 mM of TRIS/HCl.

10. A kit for isolation of nucleic acid containing the following component parts:
a) an aqueous nucleic acid-stabilising solution containing the following component parts:
a guanidinium salt; and/or
a buffer substance; and/or
a reducing agent; and/or
a detergent;
b) a composition according to one of the claims 1 to 9; and
c) a solid phase capable of bonding nucleic acid.

11. A kit according to claim 10, characterised in that the guanidinium salt in the nucleic acid-stabilising solution has been selected from guanidinium thiocyanate and guanidinium chloride.

12. A kit according to one of the claims 10 or 11, characterised in that the guanidinium salt in the nucleic acid-stabilising solution is present in a concentration of from 1 M to 8 M.

13. A kit according to one of the claims 10 to 12, characterised in that the aqueous nucleic acid-stabilising solution has a pH value of from 4 to 7.5, preferably after addition of test material, and that the buffer substance has been selected from TRIS, BEPES, MOPS, MES, citrate and phosphate buffer.

14. A kit according to one of the claims 10 to 13, characterised in that the buffer substance in the nucleic acid-stabilising solution is present in a concentration of from 10 mM to 300 mm.

15. A kit according to one of the claims 10 to 14, characterised in that the detergent in the nucleic acid-stabilising solution has been selected from Triton-X-100, NP-40, Polydocanol and Tween 20.

16. A kit according to one of the claims 10 to 15, characterised in that the detergent in the nucleic acid-stabilising solution is present in a concentration of from 5% (weight) to 30% (weight).

17. A kit according to one of the claims 10 to 16, characterised in that the reducing agent in the nucleic acid-stabilising solution has been selected from dithiothreitol, β-mercapto-ethanol and TCEP.

18. A kit according to one of the claims 10 to 17, characterised in that the reducing agent in the nucleic acid-stabilising solution is present in a concentration of from 0.1% (weight) to 10.0% (weight).

19. A kit according to one of the claims 10 to 18, characterised in that the pH of the solution to stabilise the nucleic acid lies between 4.0 and 7.5.

20. A kit according to one of the claims 10 to 19, characterised in that the nucleic acid-stabilising solution contains the following component parts:
2.5 M to 3.5 M of guanidinium thiocyanate;
40 mM to 80 mM of MES,
10% (w/v) to 20% (w/v) of Triton-X-100;
40 mM to 80 mM of DTT.

21. A kit according to one of the claims 10 to 20, characterised in that the solid phase is present separately as a fleece, filter, particle, gel, sphere, peg and/or a rod and/or is directly connected with the vessel into which the nucleic acid-containing sample is fed.

22. A kit according to one of the claims 10 to 21, characterised in that, additionally,
d) it contains a vessel into which the sample is fed.

23. A kit according to claim 22, characterised in that the vessel is a vessel for the taking of blood.

24. A vessel containing nucleic acid from a biological sample and a solution which in turn contains:
a guanidinium salt in a concentration of from 1 M to 8 M;
a detergent in a concentration of from 5% (w/v) to 25% (w/v);
a buffer in a concentration of from 100 M to 500 mM;
a reducing agent in a concentration of from 5 mM to 50 mM; and with
a pH>70.

25. A vessel according to claim 24 containing:
a guanidinium salt in a concentration of from 1.5 M to 5 M, preferably from 2.5 M to 35 M;
a detergent in a concentration of from 8% (w/v) to 20% (w/v), preferably from 10% (w/v) to 16% (w/v);
a buffer in a concentration of from 150 mM to 400 mM, preferably from 200 mM to 300 mM;
a reducing agent in a concentration of from 10 mM to 40 mM, preferably from 25 mM to 35 mM; and with
a pH>7.5, particularly preferable being ≧8.0.

26. Method for isolating a nucleic acid, comprising the following steps:
a) bringing a biological sample containing nucleic acid into contact with an aqueous solution for stabilising nucleic acid as described in claims 10 to 20;
b) addition of a composition according to one of the claims 1 to 9 to a solution according to a);
c) addition of a solid phase, capable of bonding nucleic acid, to a solution according to b);
where the sequence of the steps a), b) and c) is interchangeable.

27. Method for demonstrating the presence of a nucleic acid in a biological sample comprising the carrying out of the method according to claim 26 and detection of the isolated nucleic acid or a component part thereof.

28. Method for bonding nucleic acid to a solid phase, characterised in that the pH of the solution containing the nucleic acid and the solid phase is adjusted to a value>7.0, preferably >7.5 and particularly preferable ≧8.0.
